(19) United States
(12) Patent Application Publication
(10) Pub. No.: US 2003/0138409 A1
(43) Pub. Date: Jul. 24, 2003

(54) Th1 specific cd4 t cell lines and method for inducing them ex vivo

(76) Inventors: Veronique Pancre, Orchies (FR); Helene Gras-Masse, Merignies (FR); Ahmed Bouzidi, Annoeullin (FR); Eric Hachulla, Wattignies (FR); Claude Auriault, Nomain (FR)

Correspondence Address: BIRCH STEWART KOLASCH & BIRCH, PO BOX 747, FALLS CHURCH, VA, 22040-0747, US

(21) Appl. No.: 10/169,435

(22) PCT Filed: Dec. 28, 2000
(86) PCT No.: PCT/FR00/03708

(30) Foreign Application Priority Data
Dec. 30, 1999 (FR) 99/.16716

(51) Int. Cl. A61K 45/00; C12N 5/08

(52) U.S. Cl. 424/93.7; 435/372

(57) ABSTRACT

A TH1 specific CD4 T cell line, inducing an efficient CD8 response against an infection caused by an infectious agent, is obtained by removing from a donor a biological sample containing T cells; isolating the CD4⁺ T cells from said sample; simultaneously providing dendritic cells isolated from the same sample or another sample derived from the same donor, subjecting the previously isolated CD4⁺ T cells to in vitro immunisation with a peptide of a protein of the infectious agent exhibiting at least a T epitope, in the presence of the previously obtained dendritic cells; performing at least a restimulation, in the same conditions as for immunization, optionally substituting the dendritic cells with B cells from the same donor.

The invention relates to the production ex vivo of specific TH1 type CD4 T lymphocytes.

It more especially relates to such lymphocytes and a process for obtaining them through the ex vivo induction of specific TH1 type CD4 T lymphocyte cell lines for an immunoprophylactic or therapeutic purpose with respect to infections caused by an infective agent such as a virus, a bacterium or a parasite.

The context in which the invention is placed is explained in greater detail below with reference to infection by the Human Immunodeficiency Virus (HIV) that has engaged the efforts of numerous researchers for fifteen years or so, which means that a large amount of data is available.

Infection by HIV is characterized by chronic hyperactivity of the immune system that contrasts with a deficiency in many functions of the cells of the immune system. Ideally, vaccination against HIV should make it possible to transform the chronic infection into an acute infection, this being achieved by amplifying and accelerating the natural immune response. To date, vaccinal research on HIV has chiefly concentrated on two aspects: on one hand, the induction of a humoral response mediated by antibodies neutralizing the virus and, on the other hand, the induction of cell-mediated immunity.

Although neutralization experiments have made it possible to observe positive results in terms of protection, the exclusive use of neutralizing antibodies in a vaccinal perspective seems inapplicable due to major limitations such as restriction to minor viral isolates, as well as a need for a very high concentration of antibodies.

On the other hand, the information derived from research relating to the cell components of the anti-HIV immune response is more encouraging. Indeed, the data relating to cytotoxic activity mediated by cytotoxic suppressor T8 lymphocytes which express the CD8 marker, referred to hereinafter as CD8 T lymphocytes [Cytotoxic T Lymphocytes or CTL] show a correlation between the appearance of this activity and the control over viral infection. Cytotoxic T lymphocytes directed against sequences derived from viral proteins appear very quickly at the time of the immune response triggered by the viral infection. Generally speaking, they play an important role by eliminating the cells infected by the virus in acute infections and by blocking viral replication in persistent infections. These cytotoxic responses, as well as the induction of neutralizing antibodies, are closely linked with the so-called “T helper” (abbreviated to TH) responses mediated by the T4 lymphocytes which express the CD4 marker, referred to hereinafter as CD4 T lymphocytes. Thus, an increase in the TH response makes it possible to obtain an optimal CTL response. In particular, with individuals who control the viremia in the absence of an antiviral treatment, very strong proliferative responses of the CD4 T lymphocytes specific of the virus are observed. In all cases, it seems to be clearly established that a significant CD4 response is essential in order to obtain an efficient CD8 response.

Thus, in the framework of a vaccinal approach, it is important, henceforth, to specify the nature of the CD4 response obtained. A response of the TH1 type (IFN-γ, IL-2) or of the TH2 type (IL-4) does not have the same meaning as it might be feared, in the second case, to be more harmful than useful to vaccination, and the work accomplished hitherto as a whole clearly demonstrates the importance of IFN-γ producing TH1 cells in anti-HIV protecting immunity. However, orientation towards one type of response or the other is very difficult to control after injecting a person with an antigen, due to his genetic polymorphism.

Patent application WO 94/02156 (PCT/US93/06653) filed on Jul. 15, 1993 and which claims a priority of Jul. 16, 1992, relates to processes using dendritic cells to activate T cells. These processes are based on knowledge that was available at that time concerning the cellular part of the immune response, in particular anti-HIV.

In the present application, the authors propose a process for purifying dendritic cells for cell therapy purposes. These dentritic cells are directly pulsed with an antigen, to activate and expand specific T cells (CD4 and CD8). This protocol necessitates several isolation steps in order to obtain 80 to 90% purity of the dendritic cells.

The authors report experiments on immunization in vitro of autologous T cells by dendritic cells “exposed” to KLH (Keyhole Limpet Hemocyanine) or to SWM (Sperm Whale Myoglobin). They neither take up the question of orientation of the CD4 T response induced in these experiments, nor the question of the HLA genotype of the donor. Activation of the cells is characterized solely on the basis of a proliferative response, and the authors seem to anticipate long-term expansion (6 to 8 weeks) of the CD4 T (or CD8 T ) cells before using them in cell therapy protocols.

In the particular case of infection by HIV, the authors use a peptide of the gag protein of HIV for in vitro immunization of autologous T cells of seronegative donors. They only follow the induction of a specific CD8 response which they plan to use in the framework of adoptive cell therapy:

in the late stages of the disease, by transferring CD8 T cells from HLA-compatible healthy subjects;

in the early stages of the disease, by transferring autologous CD8 T cells (after reactivation ex vivo) or from HLA-compatible healthy subjects.

To conclude, patent application WO 94/02156, in the particular case of infection by HIV, is an approach intended to obtain a CD8 response. It does not, however, disclose the means allowing induction of a specific TH1 CD4 response (more especially generating an IFN-γ production), providing protection from infectious disorders, and whatever the HLA genotype of the donor. In addition, the processes and protocols described in this application are laborious or lengthy (purification of dentritic cells in multiple steps, expansion over 6 to 8 weeks of the CD4 or CD8 cell cultures).

The present invention remedies these shortcomings and drawbacks.

According to a first aspect, it concerns a cell line of specific CD4 T lymphocytes of the TH1 type inducing, in an efficient manner, cytotoxic T lymphocytes, i.e. an efficient CD8 response, against an infection caused by an infective agent such as, for example, a virus, a bacterium or a parasite.

According to another aspect, the invention concerns a process making it possible to induce such a cell line ex vivo in a standardized manner.

More precisely, the invention also relates to a process for the ex vivo induction of a TH1 type specific CD4 T lymphocyte cell line as defined above, which process essentially includes the steps consisting of:

a) taking from a donor a biological sample containing T lymphocytes;

b) isolating the CD4⁺ T lymphocytes from the sample taken in step a); in parallel

c) obtaining dendritic cells isolated from the same sample or from another sample originating from the same donor;

d) subjecting the CD4⁺ T lymphocytes isolated in step b) to an immunological reaction or immunization in vitro with a peptide of a protein of the infectious agent having at least one T epitope, preferably a T epitope and a B epitope, in the presence of the dentritic cells of step c);

e) carrying out at least one re-stimulation, preferably one to three re-stimulations, under the same conditions as for immunization, possibly replacing the dendritic cells with B cells from the same donor.

A “peptide of a protein of the infective agent” is to be taken here as meaning a peptide the sequence of which is identical with that of the corresponding fraction of said protein or is modified by linking a lipidic part or by inducing a chemically controlled degeneration, to an extent such that its function is unaffected, or a mixture of at least two peptides such as defined above.

According to this process, the CD4⁺ T lymphocytes, isolated in a previous step, are subjected directly to an immunological reaction or immunization in vitro with a peptide of a protein of the infective agent to be contended, in the presence of previously isolated dendritic cells.

This process can be implemented on any human or animal biological sample containing T lymphocytes. It is, a priori, of particular interest in its applications to human beings. In this case, for obvious deontological and practical reasons, the biological sample used is preferably blood and, in the case of immunoprophylactic or therapeutic applications, the blood is autologous.

The ability of this process to induce TH1 type specific CD4 T lymphocyte cell lines required for generating CTL was checked on different pathogens, as shown in the examples described below.

In the particular case of HIV, the TH1 type specific CD4 T lymphocyte cell lines required for CTL generation can be re-injected, in anti-HIV cell therapy protocols. The treated patients can, in particular, be seropositive patients in the asymptomatic phase with a CD4 T cell rate that is still normal, or patients in the process of cell renewal undergoing Highly Active AntiRetroviral Therapy (HAART). Indeed, in the latter, while a swift, marked decrease in the viremia is observed, the virus is not eradicated and, despite restoration of the CD4 T cell rate and of the overall immune response (the fight against the associated opportunistic infections), one does not see any reappearance of the specific TH1 response against the HIV that is necessary in order to eliminate said virus. The TH1 type specific CD4 T lymphocyte cell lines obtained ex vivo according to the invention can thus be used as a complement to a HAART therapy, for example a triple therapy, to restore the TH1 potential.

It goes without saying that, in the case of infections caused by other infectious agents, the protocol of use may be different. The cell lines can, in particular, be used alone, i.e. not serve as a complement to a treatment, for example in the case of vaccinating a healthy subject.

In all these applications, the TH1 type specific CD4 T lymphocyte cell lines according to the invention are autologous cells the transfer of which is capable of inducing an efficient CD8 response in vivo, without the addition# of CD8 cells.

The illustrative examples that follow are intended to explain the invention more clearly.

### EXAMPLE 1

Preparation of an Anti-HIV Specific TH1 Type CD4 T Lymphocyte Cell Line and Results

1—Preparation

In this example, the inducer is the peptide 56-68 of the Nef (Negative Factor) protein of 27 kDa, which is an HIV regulatory protein initially described as having an inhibiting effect upon viral replication in vitro. The Nef gene is to be found on 3′ side of the Env gene of HIV. References: “The HIV Nef protein: facts and hypotheses”, Guy, B. et al., Res. Virol., January-February 1992, 143(1), 34-37 and “Virological and cellular physiological roles of HIV Nef Protein”, Venkatesan, S., Res. Virol., January-February 1992, 143(1), 38-42.

The sequence of the Nef protein is described in: “Complete nucleotide sequence of AIDS virus, HTLV-III”, Ratner, L. et al., Nature 1985, 313, 277-284.

The sequence of the peptide 56-68 of the Nef protein of the HIV virus is

Ac-AWLEAQEEEEVGF-CONH₂

This peptide contains both a T epitope and a B epitope.

References: “T helper cell epitopes of the human immunodeficiency virus (HIV-1) Nef protein in rats and chimpanzees”, Estaquier et al., Mol. Immunol. April 1992, 29(4), 489-99, “Determination of B-cell epitopes of Nef HIV-1 protein: immunogenicity related to their structure”, Estaquier et al., Mol. Immunol. November 1992, 29(11), 1337-45 and “Comprehensive delineation of antigenic and immunogenic properties of peptides derived from the Nef HIV-1 regulatory protein”, Estaquier et al., Vaccine 1993, 11(11), 1083-93.

The biological medium used is peripheral human blood. The samples (100 to 150 ml) that feature in following table came from different donors typed for the Class II molecules of the major histocompatibility complex (MHC), in particular HLA-DR. The whole blood, diluted to half in phosphate buffer solution (PBS) is added on a solution for gradient specific for the isolation of mononuclear cells of human peripheral blood, Ficoll-Paquee® Amersham Pharmacia Biotech (Sweden), according to the manufacturer's recommendations, and the ring of mononuclear cells is recovered after centrifugation (400 g, 20 minutes, 20° C.).

The blood CD14⁺ monocytes are isolated by positive selection (Macs System, Milteny Biotech, Germany). The dendritic cells are then obtained with a purity of at least 80 to 90%, in a single step, by in vitro differentiation of these CD14⁺ cells placed in the presence of IL-4 (1000 U/ml) and of GM-CSF (800 U/ml) for five days in complete medium supplemented with 10% foetal calf serum (FCS). The CD14⁻ cells are, for their part, preserved in complete medium supplemented with 10% human serum of the group AB⁺, also for five days.

At the end of this five-day period, the CD4⁺ T lymphocytes are isolated by depletion or negative selection (Macs System, Milteny Biotech) from the CD14⁻ cells obtained previously.

The CD4⁺ T lymphocytes (1.10⁶/ml) are then immunized in vitro by the peptide 56-68 of the Nef protein (50 μg/ml) in the presence of the dendritic cells (DC) obtained in parallel (1.10⁶/ml).

As the inventors have succeeded in showing that deep freezing does not affect the functionality of the dendritic cells, the remaining dendritic cells are deep frozen and used for the first re-stimulation using the Nef peptide 56-68 that takes place 15 days after immunization.

For subsequent re-stimulations, the dendritic cells are, in general, replaced, in particular after the second re-restimulation, by the B cells of the donor, isolated from the same sample by depletion of the CD4⁻ cells in CD8⁺ cells (Macs system, Milteny Biotech). However, deep frozen dendritic cells can be used in all the re-stimulation procedures, as long as there are sufficient quantities thereof.

Flow diagram 1 below summarizes the different steps of isolation of the different cells to be used, starting from whole blood: dentritic cells, CD4⁺ T lymphocytes and B cells.

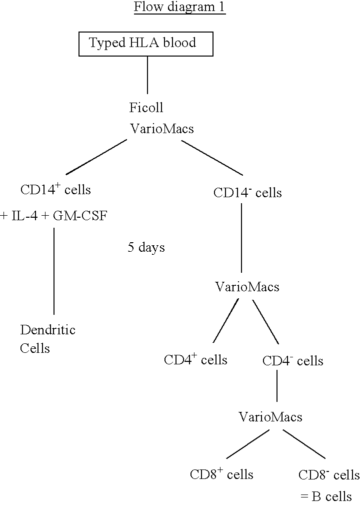

In the tests recapitulated in the following table, isolation of the specific TH1 type CD4 T lymphocytes is summarized as follows:

In vitro Immunization (I)

CD4⁺ cells+dendritic cells+peptide

Culture: 15 days

1^{st} Re-Stimulation (1S)

CD4⁺ cells+dendritic cells+peptide

Culture: 15 days

2^{nd} Re-Stimulation (2S)

CD4⁺ cells+dendritic cells or B cells+peptide

Culture: 15 days

3^{rd} Re-Stimulation (3S)

CD4⁺ cells+B cells+peptide

Culture: 15 days

2—Results

The production of cytokines and the induction of anergy (non-response) were examined.

a) Production of Cytokines.

The production of IFN-α, IL-2, IL-4 and IL-5 was searched for in the culture supernatants of the CD4⁺ T lymphocytes 24 hours and 48 hours after immunization in vitro (1) and after the successive re-stimulations (1S, 2S and 3S) by the peptide 56-68 of the Nef protein, using an ELISA method.

The results clearly show a TH 1 type orientation of the response obtained, with high production of IFN-γ and/or IL-2, but never of IL-4 or IL-5, this being the case whatever the DR genotype of the sample.

b) Induction of Anergy.

There is observed, as from the second or third re-stimulation with the peptide, depending on the case, a marked drop, or even, most often, a disappearance of IFN-γ and/or IL-2 production.

It thus seems that the optimal response is obtained after the second presentation of the peptide (1^{st} Re-Stimulation), and that repeated re-stimulations tend to anergize the CD4 T lymphocytes prepared in this example.

Under these conditions, re-injection of the cells into the patient should advantageously take place after the incubation that follows the 1^{st} re-stimulation, i.e. after about one month of culture.

The inventors have demonstrated that, after immunization in vitro with the peptide 56-68 of the Nef protein, there is very rapid generation (within 10 days) of CD4⁺ T cells having an effector memory cell phenotype producing IFN-γ after re-stimulation with the peptide 56-68 of the Nef protein. This phenotype is given on the basis of a decrease of the CD45RA markers (characterizing the naive T cells) and the CD62Ligand markers (T cell homing receptor) and of an increase of the CD45RO marker (characterizing the T memory cells) of as much as 36% at day 10.

As with the HIV infection, the appearance of CD4 memory cells specific of the virus is presently considered as a factor of a good prognostic for the evolution of the disease, this increases the advantages of using the in vitro immunization protocol according to the invention, using the peptide 56-68 of the Nef protein, for cell therapy purposes in the case of infected subjects.

It goes without saying that, for a given patient and a given protocol, different factors may intervene in obtaining the optimal response sought after, particularly as regards the moment at which it occurs. In vitro follow-up of the results is thus recommended, and even essential in each case.

TABLE OF RESULTS

| | | Number of | | | | |
|---|---|---|---|---|---|---|
| Test | | re-stimu- | Stages at which a response appears | | | |
| No. | Typing | lation | IL-4 | IL-5 | IL-2 | IFN-γ |
| 1 | DR1/IR1 | 5 | No | No | I | I, 1S, 2S, 3S |
| 2 | DR4/IR13 | 3 | No | No | I, 1S | I, 1S, 2S |
| 3 | DR3/IR4 | 2 | No | No | 1S | No |
| 4 | DR15/IR3 | 3 | No | No | No | I, 1S, 2S, 3S |
| 5 | DR3/IR11 | 1 | No | No | No | I |
| 6 | DR13/IR8 | 1 | No | No | I | I, 1S |
| 7 | DR4/IR13 | 2 | No | No | I | I, 1S, 2S |
| 8 | DR4/IR7 | 1 | No | No | I | I, 1S |
| 9 | DR3/IR11 | 2 | No | No | No | No |
| 10 | DR15/IR4 | 3 | No | No | 1S | I, 1S |
| 11 | DR15/IR13 | 3 | No | No | I, 1S, 3S | I, 1S, 2S |
| 12 | DR7/IR9 | 2 | No | No | No | I, 1S, 2S |
| 13 | DR1/IR3 | 3 | No | No | No | 1S |
| 14 | DR3/IR11 | 3 | No | No | No | I, 1S |

### EXAMPLE 2

The procedure is as for example 1, using the peptide 190-211 of Sm 28 GST (Glutathion S Transferase) of 28 kDa of the Schistosoma mansoni parasite, called Sm 28 GST 190-211, the sequence of which is:

NH₂-ENLLASSPRLAKYLSNRPATPF-COOH

After immunization in vitro and one re-stimulation, the CD4 T lymphocytes have a TH1 type orientation (with high production of IFN-γ and/or IL-2, but never of IL-4 or IL-5) of the response obtained, this being the case whatever the DR genotype of the sample.

### EXAMPLE 3

The procedure is as for example 1, using the peptide 830-846 of the tetanus toxin. This peptide is called TT 830-846. Its sequence is:

Ac-QYIKANSKFIGITELKK-CONH₂

After immunization in vitro and one re-stimulation, the CD4 T lymphocytes have a TH1 type orientation (with high production of IFN-γ and/or IL-2, but never of IL-4 or IL-5) of the response obtained, this being the case whatever the DR genotype of the sample.

### EXAMPLE 4

The procedure is as for example 1, using the peptide 307-319 of the hemaglutinine of the influenza virus. This peptide is called HA 307-319. Its sequence is:

Ac-PKYVKQNTLKLAT-CONH₂

After immunization in vitro and one re-stimulation, the CD4 T lymphocytes have a TH1 type orientation (with high production of IFN-γ and/or IL-2, but never of IL-4 or IL-5) of the response obtained, this being the case whatever the DR genotype of the sample.

Examples 2 to 4 show that the process that has been described in detail in respect of the HIV can be generalized to include infections due to other viruses, to bacteria and to parasites.

The inventors have demonstrated that, despite this generalization of the process, the T repertoire (determined on the basis of the expression of the Vbeta regions of the T receptor) expressed by the induced CD4 T cell was specific to the peptide used for immunization in vitro.

Thus, the T clones derived after immunization in vitro and one re-stimulation by peptide 56-68 of the Nef protein all have a TH1 response profile and a preferential expression of Vβ6.1. On the other hand, the clones derived, under the same conditions, with the peptide of example 3 (peptide 830-846 of the tetanus toxin) or of example 4 (peptide 307-319 of the hemaglutinine of the influenza virus) while they all have a TH1 response profile, express a different repertoire: Vβ5 for example 3 and Vβ2 for example 4, respectively, which confirms the peptide-specific nature of the induced response.

As a general conclusion, the present invention makes it possible to generate very quickly specific IFN-γ producing TH1 CD4 cells having a memory cell phenotype, this being the case whatever the HLA genotype of the donor.

Consequently, these cells can be used in cell therapy protocols, by adoptive transfer in the 10 days following immunization ex vivo, in all pathologies wherein this type of cell has been described as protective and, in particular, in infection by the HIV (in particular by the generation of specific cells of peptide 56-68 of the Nef protein).

1. A specific CD4 T lymphocyte cell line of the TH1 type inducing, in an efficient manner, cytotoxic T lymphocytes, i.e. an efficient CD8 response, against an infection caused by an infectious agent such as a virus, a bacterium or a parasite.

2. A process for the ex vivo induction of a specific CD4 T lymphocyte cell line of the TH1 type according to claim 1, which process essentially includes the steps consisting of:
a) taking from a donor a biological sample containing T lymphocytes;
b) isolating the CD4⁺ T lymphocytes from the sample taken in step a); in parallel
c) obtaining dendritic cells isolated from the same sample or from another sample originating from the same donor;
d) subjecting the CD4⁺ T lymphocytes isolated in step b) to an immunological reaction or immunization in vitro with a peptide of a protein of the infectious agent having at least one T epitope, preferably a T epitope and a B epitope, in the presence of the dentritic cells of step c);
e) carrying out at least one re-stimulation, preferably one to three re-stimulations, under the same conditions as for immunization, possibly replacing the dendritic cells with B cells from the same donor.

3. The process according to claim 2, characterized in that the biological sample is a sample of blood, preferably peripheral.

4. The process according to claim 2 or 3, characterized in that the infectious agent is HIV.

5. The process according to claim 4, characterized in that the protein is an HIV regulatory protein.

6. The process according to claim 5, characterized in that the peptide is the peptide 56-68 of Nef regulatory protein of HIV.

7. The process according to any one of claims 2 to 6, characterized in that the CD4 T lymphocytes are cultivated before the first re-stimulation and then between two successive re-stimulations for approximately fifteen days each time.

8. A specific TH1 type CD4 T lymphocyte cell line obtained from the blood of a donor and inducing, in an efficient manner, cytotoxic T lymphocytes, i.e. an efficient CD8 response, against an infection caused by an infectious agent such as a virus, a bacterium or a parasite, for the immunoprophylactic or therapeutic treatment of said donor.

9. The cell line according to claim 8, characterized in that the infectious agent is HIV.
